(19) United States
(12) Patent Application Publication
(10) Pub. No.: US 2005/0244441 A1
(43) Pub. Date: Nov. 3, 2005

(54) Cosmetic compositions

(75) Inventors: Jean-Philippe Andre Courtois, Bebington (GB); Andrew Hopkinson, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 11/115,905

(22) Filed: Apr. 27, 2005

(30) Foreign Application Priority Data
Apr. 29, 2004 (GB) 0409542.8

(51) Int. Cl. A61K 7/46; A61K 7/32

(52) U.S. Cl. 424/401; 512/1

(57) ABSTRACT

Fragranced cosmetic compositions having improved stability comprising a cosmetic active, especially an astringent antiperspirant salt dispersed in a water-immiscible carrier liquid that is gelled by a fragrance-sensitive fibre-forming gellant are obtained by selecting fragrances having a standard peak temperature depression (ΔT_{BS}) of not more than 2 K, measured by differential calorimetry, ΔT_{BS} being the difference attributable to the addition of the fragrance (blend) to a standard test composition. Especially suitable cosmetic compositions comprise an aqueous emulsion of an aluminium or aluminium/zirconium halohydrate salt optionally further complexed, dispersed in a water-immiscible carrier that is gelled by cellobiose octanonanoate.

### TECHNICAL FIELD

The present invention relates to cosmetic compositions and more particularly to cosmetic compositions comprising a gelled water-immiscible liquid containing a fragrance.

### BACKGROUND AND PRIOR ART

Cosmetic compositions are widely available in a number of different physical forms of which one popular form is based upon a gelled water-immiscible liquid that acts as a carrier for a cosmetic active ingredient. Such compositions are normally sold as sticks in which the composition is stored within a chamber having first end with an opening and a second end comprising a piston that is capable of being moved towards the first end to extrude the composition through the opening.

It is also common practice within the cosmetics industry to employ a fragrance in cosmetic compositions. There are two discernible advantages attributable to the addition of a fragrance. First, the fragrance enables the composition to smell attractively and thereby encourage purchasers of the product to use it and indeed to buy the same product again. Although many ingredients employed in cosmetic compositions are relatively odourless, some have an intrinsic malodour so that the employment of a fragrance not only imparts an attractive smell to odourless compositions, but also can mask any malodorous ingredients. Secondly, the smell from a fragranced composition often increases perceptibly when it is applied to the skin, because application increases the surface area from which the fragrance can evaporate. This confirms to the user that the composition has been applied.

It is of practical and commercial benefit if the gelled composition remains acceptably stable physically during transportation and storage of the gelled product prior to its use, which is to say remains in the form of a gel. Investigations into the preparations of gelled cosmetic compositions have shown that the presence of some fragrances in cosmetic compositions that have been gelled with various gellants that are otherwise desirable can result after a period of time in one or more of the physical properties of the gel being impaired to a detectable extent, or in other words, the composition being physically unstable. Such impaired properties can include the physical integrity of the gel being lost so that the resultant material is no longer solid, but has become a fluid mass. The physical integrity of the composition may become impaired during storage or transportation, leading for example to syneresis of carrier liquids that were gelled. The uniformity of the composition may be impaired, leading for example to the composition having a mottled appearance. Another physical property of some compositions that can be impaired comprises the translucency of such a composition. When translucency is impaired, the composition permits the transmission of less light, and at the extreme renders it opaque. Where the formulator is seeking to provide a clear product, loss of clarity represents a significant impairment.

Without being bound to any particular theory as to the cause of such physical impairment, it is believed in some cosmetic formulations to derive from an interaction between the fragrance and the gellant. Such interaction can cause or accelerate a change in the nature of the gellant, including as one possibility its crystallisation habit or size. This may itself be visible in its own right or may lead to detectable changes in the composition overall, such as in one or more of those physical properties identified above. Gellants that are more prone to interact with fragrance to lead to destabilisation of the cosmetic composition are herein referred to as being fragrance-sensitive. Other gellants which do not interact to a similar extent are not sensitive and can be distinguished by passing a stability test.

It has been recognised that at least some constituents of a composition may be encapsulated so as reduce or eliminate their interaction with the remaining constituents. Whilst this might represent an alternative means of addressing the problem of constituent interaction, it will be further recognised that a fragrance encapsulation is likely to delay its release into the atmosphere on use of the composition. As such, encapsulation of a fraction of the fragrance may complement the instant invention but is not directly equivalent to a fragrance that is not encapsulated. Encapsulation of a constituent will also at best complicate the formation of a translucent formulation and at worst render it impossible since it introduces a further particulate material which needs to be refractive index matched with the carrier liquid, a material which is likely to have a different refractive index from a dispersed active.

It is an object of the present invention to make compositions containing both a fragrance and a fragrance-sensitive gellant in which the impairment of one or more physical properties identified above is ameliorated or eliminated.

### SUMMARY OF THE INVENTION

According to the first aspect of the present invention there is provided a fragranced cosmetic composition having improved stability comprising a cosmetic active, a water-immiscible carrier for the cosmetic active, a fragrance-sensitive fibre-forming gellant for the water-immiscible carrier and a fragrance in which the fragrance has a standard peak depression, ΔT_{BS}, of not more than 2.

The present inventors have found that even when a fragrance-sensitive gellant is employed, by suitable selection of the fragrance, a comparatively stable gelled formulation can be obtained.

Herein the term “fragrance-sensitive gellant” indicates a gellant for a water-immiscible carrier having impaired stability in the presence of an oil-soluble fragrance at a concentration in the water-immiscible carrier of 2% by weight. Its stability can be tested by first gelling a water-immiscible liquid or mixture of liquids, conveniently, a 60/40 w/w mixture of hydrogenated polydecene and cyclopentadimethicone, to make a control formulation, and thereafter forming a test gel at the same concentration of gellant in the same water-immiscible liquid or mixture of liquids that additionally contains 2-phenylethanol at a concentration of 2% by weight and storing the resultant product at 45° C. The gellant is fragrance-sensitive if in 10 days storage or less, one or more of the following impairments are observed:—
the test formulation is softer than the control formulation when measured in a conventional hardness test at 22° C., one such conventional test being a sphere indentation test, and another being a needle penetration test,
or a semi-liquid phase has appeared in the test formulation or the test formulation is more liquid than the control formulation
or the test formulation has changed its appearance from prior to storage, exhibiting crystallisation or mottling to a greater extent than the control formulation.

The present invention is particularly applicable to formulations that are gelled by a fragrance-sensitive fibre-forming gellant.

When the heat flow into a sample of a gelled cosmetic formulation as specified in Table 1 hereinbelow, but without fragrance, is measured as a function of temperature by a conventional differential scanning calorimeter (e.g. a Perkin Elmer Pyris 1™ calorimeter) using a linear heating ramp at 10 K/min, the heat flow exhibits two maxima, the lower commonly between 30 and 40° C. and the higher commonly between 35 and 50° C., often between 40 and 50° C. The temperature at which the higher heat flow maximum occurs is designated T_{B} herein. The present inventors have identified that the cosmetic formulation of Table 1 containing a fragrance exhibits a peak heat flow at a temperature that is lower than when the fragrance is absent, and moreover at a temperature that differs from fragrance to fragrance. The term “peak temperature depression” is given by the equation
ΔT_{B}=T_{BN}−T_{BF}
where T_{BN} is the temperature of upper heat flow peak in the control formulation, i.e. fragrance is absent and T_{BF} is the corresponding temperature when the fragrance is present. Δ_{TB} is expressed in degrees Kelvin.

The inventors had furthermore found that there is a correlation between ΔT_{B} and the risk that the fragranced formulation will suffer a detectable impairment in stability (employing the criteria indicated hereinabove) during storage under the same conditions.

Herein by the term “standard peak depression”, or its abbreviation “ΔT_{BS}” of a fragrance is meant that the peak depression is measured at a concentration of 1% by weight of the fragrance in a composition as described in Table 1 hereinbelow.

The standard peak depression ΔT_{BS} for a fragrance is indicative of the risk that introduction of the fragrance into cosmetic compositions employing other fragrance-sensitive gellants and/other water-immiscible oils would induce instability. A ΔT_{BS} of over 2 indicates a higher risk and of below 2 indicates a lower risk. It will be recognised that it is the extent of standard peak depression that is important and not the actual temperature at which peak heat flow into the composition occurs, the latter being likely to vary, for example, depending on the particular gellant mixture of water-immiscible oils employed. Unless otherwise indicated, the invention relates to formulations contains fragrance that is not encapsulated. The fragrance may consist of a single fragrance component having an appropriate ΔT_{BS}, but normally the fragrance consists of a mixture of at least two fragrance components and typically from at least 5, especially at least 10 components, the resultant mixture having the appropriate ΔT_{B} S of not more than 2.

### DETAILED DESCRIPTION OF THE INVENTION

The instant invention relates to the selection of fragrances for incorporation into cosmetic compositions comprising a cosmetic active, a carrier and a fragrant-sensitive gellant for the carrier on the basis of the extent to which the fragrance depresses the higher temperature in a standard reference system at which the maximum heat flow into the carrier/gellant mixture occurs compared with when the fragrance is absent (ΔT_{BS}) when measured under the standard conditions defined above.

The change ΔT_{BS} herein is measured using the same conventional differential scanning calorimeter for both the fragrance-containing and fragrance-absent control compositions, and in particular the Perkin Elmer Pyris 1 TM calorimeter.

It is particularly convenient to employ fragrance blends which cause a standard temperature depression ΔT_{BS} when incorporated into the reference carrier/gellant composition of from 1 to 2. A number of fragrances or blends have ΔT_{B} S of from 1.1 to 1.5 and others from 1.5 to 1.9. It is often of practical benefit to select a fragrance having ΔT_{BS} of not higher than 1.5.

The standard depression ΔT_{BS} can be measured for the actual fragrance blend that is intended to be incorporated. That is one way to be certain about whether or not ΔT_{BS} is according to the invention or is too high. However, if the user chooses at least initially not to make the measurement using the complete fragrance blend, an approximation can be made using individual measurements of the principle weight components or intermediate combinations of components of the fragrance blend, weight averaged, each once again measured at an incorporation of 1 part by weight in the reference system. As a reasonably close approximation, the ΔT_{BS} from the eventual full fragrance corresponds to the weight average of the ΔT_{BS} of the individual components. This is a convenient guide which can subsequently be checked with the full fragrance.

It will be recognised that the need for the full fragrance to satisfy the specified test condition herein does not preclude the formulator from employing any particular fragrance component, even those which if used alone would cause the peak temperature depression ΔT_{BS} of more than 2, but instead constrains the proportion of such components which can be incorporated and requires the presence of enough components causing a lower temperature depression to act as a counterbalance and bring the weighted average of the fragrance blend ΔT_{BS} to not more than 2, and conveniently from 1 to 2.

Accordingly, the fragrance can comprise any of the following fragrance components even if it has a high ΔT_{BS} subject if need be to the presence of a counterbalancing proportion of a component or mixture of components for a component causing low ΔT_{BS}.

| | |
|---|---|
| Abies Alba Leaf Oil | Linalyl Acetate |
| Abies Balsamea (Balsam Canada) | Linoleic Acid |
| Resin | |
| Abies Pectinata Oil | Linolenic Acid |
| Abies Sibirica Oil | Linum Usitatissimum (Linseed) |
| | Seed Oil |
| Acacia Catechu Gum | Lippia Citriodora Flower Water |
| Acacia Senegal Gum | Liquidambar Styraciflua Oil |
| Acetaldehyde | Litsea Cubeba Fruit Oil |
| Acetanilid | Longifolene |
| Acetic Acid | Lysine |
| 1-Acetonaphthone | Maleic Acid |
| 2-Acetonaphthone | Malic Acid |
| Acetone | Malonic Acid |
| Acetyl Hexamethyl Indan | Marrubium Vulgare Extract |
| Acetyl Hexamethyl Tetralin | Massoy Bark Oil |
| Acetyl Tributyl Citrate | Medicago Sativa (Alfalfa) |
| | Extract |
| Acetyl Triethyl Citrate | MEK |
| Achillea Millefolium Extract | Melaleuca Alternifolia (Tea |
| | Tree) Leaf Oil |
| Achillea Millefolium Oil | Melaleuca Ericifolia Leaf Oil |
| Actinidia Chinensis (Kiwi) | Melaleuca Leucadendron Cajaput |
| Fruit Water | Oil |
| Adipic Acid | Melilotus Officinalis Extract |
| Agar | Melissa Officinalis Leaf |
| | Extract |
| Agropyron Repens Root Extract | Melissa Officinalis Leaf Oil |
| Alanine | Mentha Arvensis Extract |
| Albizia Julibrissin Bark | Mentha Arvensis Leaf Oil |
| Extract | |
| Alcohol | Mentha Arvensis Powder |
| Alcohol Denat. | p-Menthan-7-ol |
| Algae Extract | Mentha Piperita (Peppermint) |
| | Leaf |
| Algin | Mentha Piperita (Peppermint) |
| | Leaf Extract |
| Allyl Caproate | Mentha Piperita (Peppermint) |
| | Leaf Water |
| Aloe Barbadensis Leaf | Mentha Piperita (Peppermint) |
| | Oil |
| Aloe Barbadensis Leaf Water | Mentha Pulegium Oil |
| Amidinoproline | Mentha Viridis (Spearmint) |
| | Extract |
| Aminomethyl Propanediol | Mentha Viridis (Spearmint) |
| | Leaf Oil |
| Ammonia | Menthol |
| Ammonium Chloride | Menthone Glycerin Acetal |
| Ammonium Glycyrrhizate | Menthoxypropanediol |
| Amyl Acetate | Menthyl Acetate |
| Amyl Benzoate | Menthyl Lactate |
| Amyl Cinnamal | Menthyl Salicylate |
| Amylcinnamyl Alcohol | Methionine |
| Amyl Salicylate | Methoxydiglycol |
| Amyris Balsamifera Bark Oil | Methoxyethanol |
| Anethole | Methoxyethanol Acetate |
| Angelica Archangelica Root | Methoxyindane |
| Extract | |
| Angelica Archangelica Root Oil | Methoxyisopropanol |
| Aniba Rosaeodora (Rosewood) | Methyl Acetate |
| Wood Extract | |
| Aniba Rosaeodora (Rosewood) | p-Methyl Acetophenone |
| Wood Oil | |
| Anisaldehyde | Methylal |
| Anise Alcohol | Methyl Alcohol |
| p-Anisic Acid | Methyl Anthranilate |
| Anthemis Nobilis Flower | 4-Methylbenzaldehyde |
| Extract | |
| Anthemis Nobilis Flower Oil | Methyl Benzoate |
| Apium Graveolens (Celery) | Methyl Benzodioxepinone |
| Extract | |
| Arginine | Methyl 4-t-Butylbenzoate |
| Arnica Montana Flower Extract | Methyl Caproate |
| Artemisia Annua Extract | Methyl Caprylate |
| Artemisia Princeps Leaf Water | Methylcellulose |
| Artemisia Tridentata Oil | 6-Methyl Coumarin |
| Ascorbic Acid | Methyldihydrojasmonate |
| Ascorbyl Palmitate | Methyl Diisopropyl |
| | Propionamide |
| Asparagine | Methyl Eugenol |
| Aspartic Acid | Methyl Hexyl Ether |
| Astragalus Gummifer Gum | Methyl Hydrogenated Rosinate |
| Azelaic Acid | Methyl Lactate |
| Backhousia Citriodora Oil | Methyl Lactic Acid |
| Beeswax | Methyl Laurate |
| Benzaldehyde | Methyl Linoleate |
| Benzoic Acid | Methyl 3-Methylresorcylate |
| Benzophenone | Methyl Myristate |
| Benzophenone-2 | Methyl Nicotinate |
| Benzophenone-6 | Methyl 2-Octynoate |
| Benzyl Acetate | Methyl Oleate |
| Benzyl Alcohol | Methyl Palmitate |
| Benzyl Benzoate | Methylparaben |
| Benzyl Benzoyloxybenzoate | Methyl Pelargonate |
| Benzyl Cinnamate | Methyl Phenylbutanol |
| 3-Benzylidene Camphor | Methyl Rosinate |
| Benzyl Laurate | Methyl Salicylate |
| Benzyl Salicylate | Methyl Stearate |
| Betula Alba Bark Extract | MIBK |
| Betula Alba Extract | Michelia Alba Flower Oil |
| Betula Alba Leaf Extract | Michelia Alba Leaf Oil |
| Betula Alba Oil | Michelia Champaca Oil |
| BHA | Mimosa Tenuiflora Bark Extract |
| BHT | Mimosa Tenuiflora Leaf Extract |
| Bisabolol | Mineral Oil |
| Bixa Orellana Extract | Mixed Cresols |
| Bixa Orellana Seed Extract | Mixed Ionones |
| Boesenbergia Pandurata Root | Monarda Didyma Oil |
| Oil | |
| Borneol | Musa Sapientum (Banana) Flower |
| | Water |
| Boswellia Carterii Extract | Musk Ketone |
| Boswellia Carterii Oil | Myrcenol |
| Boswellia Serrata Gum | Myrica Gale Extract |
| Boswellia Serrata Oil | Myristic Acid |
| 2,3-Butanediol | Mentha Viridis (Spearmint) |
| | Extract |
| Butoxydiglycol | Mentha Viridis (Spearmint) |
| | Leaf Oil |
| Butoxyethanol | Menthol |
| Butoxyethyl Acetate | Menthone Glycerin Acetal |
| Butyl Acetate | Menthoxypropanediol |
| n-Butyl Alcohol | Menthyl Acetate |
| t-Butyl Alcohol | Menthyl Lactate |
| 4-t-Butyl Benzaldehyde | Menthyl Salicylate |
| Butyl Benzoate | Methionine |
| 4-t-Butylbenzoic Acid | Methoxydiglycol |
| 2-t-Butylcyclohexyl Acetate | Methoxyethanol |
| 2-t-Butylcyclohexyloxybutanol | Methoxyethanol Acetate |
| Butylene Glycol | Methoxyindane |
| Butyl Lactate | Methoxyisopropanol |
| Butyl Methacrylate | Methyl Acetate |
| Butyl Oleate | p-Methyl Acetophenone |
| Butylparaben | Methylal |
| Butylphenyl Methylpropional | Methyl Alcohol |
| Butyl Stearate | Methyl Anthranilate |
| Butyric Acid | 4-Methylbenzaldehyde |
| Butyrolactone | Methyl Benzoate |
| Caffeic Acid | Methyl Benzodioxepinone |
| Caffeine | Methyl 4-t-Butylbenzoate |
| Calcium Acetate | Methyl Caproate |
| Calcium Alginate | Methyl Caprylate |
| Calendula Officinalis Flower | Methylcellulose |
| Extract | |
| Calendula Officinalis Flower | 6-Methyl Coumarin |
| Oil | |
| Callitris Introtropica Wood | Methyldihydrojasmonate |
| Oil | |
| Callitris Quadrivalvis Gum | Methyl Diisopropyl |
| | Propionamide |
| Camellia Oleifera Leaf | Methyl Eugenol |
| Camellia Sinensis Leaf Extract | Methyl Hexyl Ether |
| Camellia Sinensis Leaf Water | Methyl Hydrogenated Rosinate |
| Camphene | Methyl Lactate |
| Camphor | Methyl Lactic Acid |
| Camphylcyclohexanol | Methyl Laurate |
| Cananga Odorata Flower Oil | Methyl Linoleate |
| Cananga Odorata Flower Wax | Methyl 3-Methylresorcylate |
| Canarium Commune Gum Oil | Methyl Myristate |
| Canarium Luzonicum Gum | Methyl Nicotinate |
| Nonvolatiles | |
| Capric Acid | Methyl 2-Octynoate |
| Caproic Acid | Methyl Oleate |
| Caprylic Acid | Methyl Palmitate |
| Caprylic Alcohol | Methylparaben |
| Caprylic/Capric Triglyceride | Methyl Pelargonate |
| Caprylyl Butyrate | Methyl Phenylbutanol |
| Capsaicin | Methyl Rosinate |
| Capsicum Annuum Extract | Methyl Salicylate. |
| Capsicum Frutescens Resin | Methyl Stearate |
| Caramel | MIBK |
| Carmine | Michelia Alba Flower Oil |
| Carthamus Tinctorius | Michelia Alba Leaf Oil |
| (Safflower) Seed Oil | |
| Carum Carvi (Caraway) Fruit | Michelia Champaca Oil |
| Oil | |
| Carum Carvi (Caraway) Seed | Mimosa Tenuiflora Bark Extract |
| Extract | |
| Carum Carvi (Caraway) Seed Oil | Mimosa Tenuiflora Leaf Extract |
| Carum Petroselinum (Parsley) | Mineral Oil |
| Seed Oil | |
| Carvone | Mixed Cresols |
| Beta-Caryophyllene | Mixed Ionones |
| Cedrol | Monarda Didyma Oil |
| Cedrus Atlantica (Cedarwood) | Musa Sapientum (Banana) Flower |
| Bark Oil | Water |
| Cedrus Deodara Wood Oil | Musk Ketone |
| Cellulose Gum | Myrcenol |
| Ceratonia Siliqua (Carob) | Myrica Gale Extract |
| Fruit Extract | |
| Ceratonia Siliqua Gum | Myristic Acid |
| Cetyl Acetate | Myristica Fragrans (Nutmeg) |
| | Kernel Oil |
| Cetyl Alcohol | Myristyl Alcohol |
| Cetyl Palmitate | Myrocarpus Fastigiatus Oil |
| Chamaecyparis Obtusa Oil | Myroxylon Balsamum (Balsam |
| | Tolu) Resin |
| Chamaecyparis Obtusa Water | Myroxylon Pereirae (Balsam |
| | Peru) Oil |
| Chamomilla Recutita | Myroxylon Pereirae (Balsam |
| (Matricaria) Flower Extract | Peru) Resin |
| Chamomilla Recutita | Myrrhis Odorata Extract |
| (Matricaria) Flower Oil | |
| Chamomilla Recutita | Myrtus Communis Leaf Water |
| (Matricaria) Flower Water | |
| Chamomilla Recutita | Myrtus Communis Oil |
| (Matricaria) Leaf Extract | |
| Chondrus Crispus (Carrageenan) | 2-Naphthol |
| Chouji Yu | Narcissus Poeticus Extract |
| Cichorium Intybus (Chicory) | Narcissus Poeticus Flower Wax |
| Leaf Extract | |
| Cichorium Intybus (Chicory) | Nasturtium Officinale Extract |
| Root Extract | |
| Cinnamal | Nelumbium Speciosum Flower |
| | Water |
| Cinnamomum Camphora (Camphor) | Nelumbo Nucifera Flower Water |
| Bark Oil | |
| Cinnamomum Camphora (Camphor) | Neohesperidin Dihydrochalcone |
| Leaf Extract | |
| Cinnamomum Cassia Leaf Oil | Nepeta Cataria Extract |
| Cinnamomum Zeylanicum Bark Oil | Nicotiana Tabacum (Tobacco) |
| | Leaf Extract |
| Cinnamyl Acetate | Nindou Ekisu |
| Cinnamyl Alcohol | Nitrous Oxide |
| Cistus Ladaniferus Extract | Gamma-Nonalactone |
| Cistus Ladaniferus Oil | Nonyl Acetate |
| Cistus Ladaniferus Resin | Nopyl Acetate |
| Cistus Monspeliensis Extract | Ocimum Basilicum (Basil) |
| | Extract |
| Citral | Ocimum Basilicum (Basil) Oil |
| Citric Acid | Octadecane |
| Citronellal | Octyldodecanol |
| Citronellol | Olax Dissitiflora Root Oil |
| Citronellyl Acetate | Olea Europaea (Olive) Fruit |
| | Oil |
| Citronellyl Methylcrotonate | Oleic Acid |
| Citrus Aurantifolia (Lime) Oil | Oleth-2 |
| Citrus Aurantium Amara (Bitter | Oleth-3 |
| Orange) Flower Water | |
| Citrus Aurantium Amara (Bitter | Oleth-4 |
| Orange) Oil | |
| Citrus Aurantium Amara (Bitter | Oleth-5 |
| Orange) Peel Extract | |
| Citrus Aurantium Bergamia | Oleth-6 |
| (Bergamot) Fruit Oil | |
| Citrus Aurantium Dulcis | Oleth-7 |
| (Orange) Flower Oil | |
| Citrus Aurantium Dulcis | Oleth-8 |
| (Orange) Flower Water | |
| Citrus Aurantium Dulcis | Oleth-9 |
| (Orange) Fruit Extract | |
| Citrus Aurantium Dulcis | Oleth-10 |
| (Orange) Fruit Water | |
| Citrus Aurantium Dulcis | Oleth-11 |
| (Orange) Oil | |
| Citrus Grandis (Grapefruit) | Oleth-12 |
| Fruit Water | |
| Citrus Grandis (Grapefruit) | Oleth-15 |
| Peel Oil | |
| Citrus Grandis/Paradisi Fruit | Oleth-16 |
| Water | |
| Citrus Limon Leaf Oil | Oleth-20 |
| Citrus Medica Limonum (Lemon) | Oleth-23 |
| Fruit Extract | |
| Citrus Medica Limonum (Lemon) | Oleth-24 |
| Fruit Water | |
| Citrus Medica Limonum (Lemon) | Oleth-25 |
| Peel Oil | |
| Citrus Medica Vulgaris Peel | Oleth-30 |
| Oil | |
| Citrus Nobilis (Mandarin | Oleth-35 |
| Orange) Fruit Extract | |
| Citrus Nobilis (Mandarin | Oleth-40 |
| Orange) Peel Extract | |
| Citrus Nobilis (Mandarin | Oleth-44 |
| Orange) Peel Oil | |
| Citrus Reticulata Leaf Oil | Oleth-50 |
| Citrus Tangerina (Tangerine) | Oleth-82 |
| Peel Oil | |
| Citrus Unshiu Peel Powder | Oleth-106 |
| CI 75470 | Oleyl Alcohol |
| Cnidium Officinale Root Water | Olibanum |
| Cochlearia Armoracia | Opoponax Oil |
| (Horseradish) Root Extract | |
| Cocos Nucifera (Coconut) Oil | Orange Yu |
| Cocos Nucifera (Coconut) Water | Origanum Majorana Leaf Extract |
| Cod Liver Oil | Origanum Majorana Leaf Oil |
| Coffea Arabica (Coffee) | Origanum Vulgare Flower |
| Extract | Extract |
| Coffea Arabica (Coffee) Seed | Ormenis Multicaulis Extract |
| Extract | |
| Coffea Arabica (Coffee) Seed | Ormenis Multicaulis Oil |
| Oil | |
| Coleus Forskohlii Root Oil | Oryza Sativa (Rice) Bran Water |
| Commiphora Myrrha Oil | Osmanthus Fragrans Flower |
| | Extract |
| Commiphora Myrrha Resin | Palmitic Acid |
| Copaifera Officinalis (Balsam | Panax Ginseng Root Water |
| Copaiba) Resin | |
| Coriandrum Sativum (Coriander) | Pandanus Amaryllifolius Leaf |
| Extract | Extract |
| Coriandrum Sativum (Coriander) | Paraffin |
| Fruit Oil | |
| Corylus Avellana (Hazel) Leaf | PEG-2 Hydrogenated Castor Oil |
| Water | |
| Corylus Avellana (Hazel) Seed | PEG-5 Hydrogenated Castor Oil |
| Oil | |
| Coumarin | PEG-6 Hydrogenated Castor Oil |
| m-Cresol | PEG-7 Hydrogenated Castor Oil |
| o-Cresol | PEG-10 Hydrogenated Castor Oil |
| p-Cresol | PEG-16 Hydrogenated Castor Oil |
| Crocus Sativus Flower Extract | PEG-20 Hydrogenated Castor Oil |
| Crotonaldehyde | PEG-25 Hydrogenated Castor Oil |
| Crotonic Acid | PEG-30 Hydrogenated Castor Oil |
| Cryptocarya Crassinervia Bark | PEG-35 Hydrogenated Castor Oil |
| Oil | |
| Cryptocarya Massoy Bark | PEG-40 Hydrogenated Castor Oil |
| Extract | |
| Cuminum Cyminum (Cumin) Seed | PEG-45 Hydrogenated Castor Oil |
| Extract | |
| Cuminum Cyminum (Cumin) Seed | PEG-50 Hydrogenated Castor Oil |
| Oil | |
| Cupressus Sempervirens Oil | PEG-54 Hydrogenated Castor Oil |
| Curcuma Longa (Turmeric) Root | PEG-55 Hydrogenated Castor Oil |
| Extract | |
| Cyamopsis Tetragonoloba (Guar) | PEG-60 Hydrogenated Castor Oil |
| Gum | |
| Cyclamen Aldehyde | PEG-80 Hydrogenated Castor Oil |
| Cyclopentadecanone | PEG-100 Hydrogenated Castor |
| | Oil |
| Cymbopogon Flexuosus Oil | PEG-200 Hydrogenated Castor |
| | Oil |
| Cymbopogon Martini Oil | PEG-10 Sorbitan Laurate |
| Cymbopogon Nardus (Citronella) | PEG-40 Sorbitan Laurate |
| Oil | |
| Cymbopogon Schoenanthus Oil | PEG-44 Sorbitan Laurate |
| p-Cymene | PEG-75 Sorbitan Laurate |
| Cyperus Esculentus Root Oil | PEG-80 Sorbitan Laurate |
| Cysteine | PEG-3 Sorbitan Oleate |
| Cystine | PEG-6 Sorbitan Oleate |
| Cytisus Scoparius Flower | PEG-3 Sorbitan Stearate |
| Extract | |
| Dalea Spinosa Seed Oil | PEG-4 Sorbitan Stearate |
| Alpha-Damascone | PEG-6 Sorbitan Stearate |
| Daucus Carota Sativa (Carrot) | PEG-40 Sorbitan Stearate |
| Root Water | |
| Daucus Carota Sativa (Carrot) | PEG-60 Sorbitan Stearate |
| Seed Oil | |
| Delta-Decalactone | Pelargonic Acid |
| Decanal | Pelargonium Graveolens Extract |
| Decane | Pelargonium Graveolens Flower |
| | Oil |
| Decenal | Pelargonium Graveolens Wax |
| Decyl Alcohol | Pentadecalactone |
| Denatonium Benzoate | Pentadecyl Alcohol |
| Diacetone Alcohol | Perillaldehyde |
| Dianthus Caryophyllus Flower | Persea Gratissima (Avocado) |
| Oil | Fruit Water |
| Dibutyl Phthalate | Phenethyl Acetate |
| Dibutyl Sebacate | Phenethyl Alcohol |
| Diethyl Adipate | Phenol |
| Diethylamine | Phenoxyethanol |
| Diethyl Caprylamide | Phenylalanine |
| Diethylene Glycol | Phenyl Benzoate |
| Diethylhexyl Phthalate | Phenylisohexanol |
| Diethylhexyl Sebacate | Phenylmethylpentanal |
| Diethyl Oxalate | o-Phenylphenol |
| Diethyl Phthalate | Phenylpropanol |
| Diethyl Sebacate | Phenyl Salicylate |
| Diethyl Succinate | Phosphoric Acid |
| Dihydrocitronellol | Phytol |
| Dihydrocoumarin | Picea Excelsa Leaf Oil |
| Dihydrojasmonate | Pimenta Acris (Bay) Leaf Oil |
| Dihydroxyindole | Pimpinella Anisum (Anise) |
| | Fruit Extract |
| Diisobutyl Adipate | Pinus Palustris Oil |
| Diisopropyl Adipate | Pinus Palustris Tar Oil |
| Dimethyl Brassylate | Pinus Pumilio Oil |
| Dimethyl Carbonate | Pinus Strobus Cone Extract |
| 2,4-Dimethyl-3-Cyclohexene | Pinus Sylvestris Cone Extract |
| Carboxaldehyde | |
| Dimethyl Hexahydronaphthyl | Pinus Sylvestris Cone Oil |
| Dihydroxymethyl Acetal | |
| Dimethylhydroxy Furanone | Pinus Sylvestris Leaf Oil |
| Dimethyloctahydro-2- | Piper Betle Leaf Oil |
| Naphthaldehyde | |
| 2,6-Dimethyl-7-Octen-2-ol | Piper Nigrum (Black Pepper) |
| | Fruit Oil |
| Dimethyl Phenylpropanol | Pistacia Lentiscus (Mastic) |
| | Gum |
| Dimethyl Phthalate | Pogostemon Cablin Oil |
| Dimethyl Succinate | Polianthes Tuberosa Extract |
| Dipentene | Polysorbate 20 |
| Diphenyl Methane | Polysorbate 21 |
| Dipropylene Glycol | Polysorbate 60 |
| Dipteryx Odorata Seed Extract | Polysorbate 61 |
| Disodium Phosphate | Polysorbate 80 |
| Disodium Succinate | Polysorbate 81 |
| Dodecene | Pongamol |
| Eicosane | Potassium Acetate |
| Elettaria Cardamomum Seed | Potassium Sorbate |
| Extract | |
| Elettaria Cardamomum Seed Oil | PPG-2-Buteth-2 |
| Erythorbic Acid | PPG-2-Buteth-3 |
| Ethoxydiglycol | PPG-3-Buteth-5 |
| Ethoxyethanol | PPG-4-Buteth-4 |
| Ethoxyethanol Acetate | PPG-5-Buteth-5 |
| Ethyl Acetate | PPG-5-Buteth-7 |
| Ethyl Benzoate | PPG-7-Buteth-4 |
| Ethyl Butyl Valerolactone | PPG-7-Buteth-10 |
| Ethylcellulose | PPG-9-Buteth-12 |
| Ethyl Cinnamate | PPG-10-Buteth-9 |
| Ethyl Cyclohexyl Propionate | PPG-12-Buteth-12 |
| Ethyl 2,2- | PPG-12-Buteth-16 |
| Dimethylhydrocinnamal | |
| Ethylene Brassylate | PPG-15-Buteth-20 |
| Ethylene Dodecanedioate | PPG-17-Buteth-17 |
| Ethyl Ether | PPG-19-Buteth-19 |
| Ethyl Hexanediol | PPG-20-Buteth-30 |
| Ethylhexyl Ethylhexanoate | PPG-24-Buteth-27 |
| Ethylhexyl Palmitate | PPG-26-Buteth-26 |
| Ethylhexyl Salicylate | PPG-28-Buteth-35 |
| Ethyl Lactate | PPG-30-Buteth-30 |
| Ethyl Laurate | PPG-33-Buteth-45 |
| Ethyl Linoleate | PPG-36-Buteth-36 |
| Ethyl Linolenate | PPG-38-Buteth-37 |
| Ethyl Menthane Carboxamide | PPG-2 Methyl Ether |
| Ethyl Methacrylate | Proline |
| Ethyl Methylphenylglycidate | Propionic Acid |
| Ethyl Myristate | Propyl Acetate |
| Ethyl Nicotinate | Propyl Alcohol |
| Ethyl Oleate | Propyl Benzoate |
| Ethyl Palmitate | Propylene Glycol |
| Ethylparaben | Propylene Glycol Alginate |
| Ethyl Pelargonate | Propylene Glycol Butyl Ether |
| Ethyl Phenethyl Acetal | Propylene Glycol Stearate |
| Ethyl Phenylacetate | Propyl Gallate |
| Ethyl Pyruvate | Propylparaben |
| Ethyl Ricinoleate | Prunus Amygdalus Amara (Bitter |
| | Almond) Kernel Oil |
| Ethyl Stearate | Prunus Amygdalus Dulcis (Sweet |
| | Almond) Oil |
| Ethyl Thioglycolate | Prunus Armeniaca (Apricot) |
| | Fruit Water |
| Ethyl Trimethylcyclopentene | Prunus Armeniaca (Apricot) |
| Butenol | Kernel Oil |
| Ethyl Vanillin | Prunus Cerasus (Bitter Cherry) |
| | Seed Oil |
| Eucalyptol | Prunus Serotina (Wild Cherry) |
| | Bark Extract |
| Eucalyptus Citriodora Oil | Prunus Serotina (Wild Cherry) |
| | Fruit Extract |
| Eucalyptus Globulus Leaf Oil | Punica Granatum Bark Extract |
| Eucalyptus Globulus Leaf Water | Punica Granatum Extract |
| Eugenia Caryophyllus (Clove) | Pyrocatechol |
| Flower Extract | |
| Eugenia Caryophyllus (Clove) | Pyrogallol |
| Flower Oil | |
| Eugenia Caryophyllus (Clove) | Pyrus Cydonia Seed Extract |
| Leaf Oil | |
| Eugenol | Pyrus Malus (Apple) Fruit |
| | Water |
| Eugenyl Acetate | Pyruvic Acid |
| Euphorbia Cerifera | Quillaja Saponaria Bark |
| (Candelilla) Wax | Extract |
| Evernia Furfuracea (Treemoss) | Quillaja Saponaria Root |
| Extract | Extract |
| Evernia Prunastri (Oakmoss) | Quinine |
| Extract | |
| Farnesene | Raspberry Ketone |
| Farnesol | Raspberryketone Glucoside |
| Farnesyl Acetate | Resorcinol |
| Ferula Galbaniflua (Galbanum) | Rhamnose |
| Resin Oil | |
| Ficus Carica (Fig) Fruit Water | Rhododendron Chrysanthum Leaf |
| | Extract |
| Foeniculum Vulgare (Fennel) | Rhododendron Ferrugineum |
| Fruit Extract | Extract |
| Foeniculum Vulgare (Fennel) | Ricinus Communis (Castor) Seed |
| Fruit Powder | Oil |
| Foeniculum Vulgare (Fennel) | Rosa Canina Flower |
| Oil | |
| Foeniculum Vulgare (Fennel) | Rosa Centifolia Flower Extract |
| Water | |
| Formic Acid | Rosa Centifolia Flower Oil |
| Fucus Vesiculosus Extract | Rosa Damascena Extract |
| Fumaric Acid | Rosa Damascena Flower Oil |
| Furfural | Rosa Damascena Flower Water |
| Fusanus Spicatus Wood Oil | Rosa Damascena Flower Wax |
| Galactoarabinan | Rosa Eglentaria Extract |
| Gardenia Florida Oil | Rosa Moschata Oil |
| Gaultheria Procumbens | Rosa Multiflora Fruit Extract |
| (Wintergreen) Leaf Extract | |
| Gaultheria Procumbens | Rose Flower Oil |
| (Wintergreen) Leaf Oil | |
| Gentiana Lutea Root Extract | Rosmarinus Officinalis |
| | (Rosemary) Flower Wax |
| Geraniol | Rosmarinus Officinalis |
| | (Rosemary) Leaf Extract |
| Geranium Maculatum Oil | Rosmarinus Officinalis |
| | (Rosemary) Leaf Oil |
| Geranyl Acetate | Rosmarinus Officinalis |
| | (Rosemary) Leaf Water |
| Gluconic Acid | Rubus Fruticosus (Blackberry) |
| | Fruit Extract |
| Gluconolactone | Rubus Fruticosus (Blackberry) |
| | Leaf Extract |
| Glucose Pentaacetate | Rubus Idaeus (Raspberry) Fruit |
| | Water |
| Glutamic Acid | Rubus Idaeus (Raspberry) Leaf |
| | Wax |
| Glutamine | Ruta Graveolens (Rue) Oil |
| Glutaral | Saccharin |
| Glutaric Acid | Salicylic Acid |
| Glutathione | Salvia Lavandulaefolia Oil |
| Glycerin | Salvia Officinalis (Sage) Leaf |
| | Water |
| Glyceryl Oleate | Salvia Officinalis (Sage) Oil |
| Glyceryl Rosinate | Salvia Sclarea (Clary) Oil |
| Glyceryl Stearate | Sambucus Nigra Oil |
| Glycine | Sambucus Nigra Wax |
| Glycine Soja (Soybean) Oil | Santalum Album (Sandalwood) |
| | Oil |
| Glycol | Sassafras Officinale Root Oil |
| Glycyrrhiza Glabra (Licorice) | Satureia Hortensis Extract |
| Glycyrrhizic Acid | Schinus Molle Oil |
| Glyoxal | Sclareolide |
| Gnaphalium Leontopodium Water | Serine |
| Gossypium Herbaceum (Cotton) | Sesamum Indicum (Sesame) Seed |
| Fruit Water | Oil |
| Guaiazulene | Sisymbrium Irio Seed Oil |
| Hakka Yu | Beta-Sitosterol |
| Hay Water | Sodium Acetate |
| Helichrysum Stoechas Extract | Sodium Benzoate |
| Heliotropine | Sodium Citrate |
| 2-Heptylcyclopentanone | Sodium Glutamate |
| Hexadecanolactone | Sodium Hexametaphosphate |
| Hexamethylindanopyran | Sodium Saccharin |
| Hexanal | Solanum Lycopersicum (Tomato) |
| | Fruit Water |
| 3-Hexenol | Sorbic Acid |
| Hexyl Alcohol | Sorbitan Oleate |
| Hexyl Cinnamal | Sorbitan Stearate |
| Hexylene Glycol | Sorbitol |
| Hibiscus Abelmoschuus Extract | Spartium Junceum Flower |
| | Extract |
| Hinokitiol | Stearic Acid |
| Hippuric Acid | Stearyl Alcohol |
| Histidine | Sterculia Urens Gum |
| Homosalate | Styrax Benzoin Gum |
| Humulus Lupulus (Hops) Cone | Styrax Benzoin Resin Extract |
| Oil | |
| Humulus Lupulus (Hops) Extract | Succinic Acid |
| Hydroabietyl Alcohol | Sucrose Octaacetate |
| Hydrogenated | Synthetic Wax |
| Ethylbicycloheptane Guaiacol | |
| Hydrogenated Lanolin | Tagetes Minuta Flower Oil |
| Hydrogenated Polydecene | Taimu Yu |
| Hydrogenated Rosin | Tanacetum Cinerariifolium |
| | (Pyrethrum) Flower Extract |
| Hydroquinone | Tannic Acid |
| p-Hydroxyanisole | Tarchonanthus Camphoratus Oil |
| 4-Hydroxybenzoic Acid | Tar Oil |
| Hydroxycitronellal | Tartaric Acid |
| Hydroxyisohexyl 3-Cyclohexene | Taurine |
| Carboxaldehyde | |
| Hydroxymethoxybenzyl | TBHQ |
| Pelargonamide | |
| Hyptis Suaveolens Seed Oil | Terpineol |
| Hyssopus Officinalis Extract | 4-Terpineol |
| Hyssopus Officinalis Leaf Oil | Terpineol Acetate |
| Illicium Verum (Anise) Oil | Tetrahydrofurfuryl Acetate |
| Iris Florentina Root Extract | Tetrahydrofurfuryl Alcohol |
| Isoamyl Acetate | Tetramethyl Cyclopentene |
| | Butenol |
| Isoamyl Alcohol | Theobroma Cacao (Cocoa) Seed |
| | Butter |
| Isoamyl Cinnamate | Theobromine |
| Isoamyl Laurate | Thiamine HCl |
| Isobornyl Acetate | Thiolactic Acid |
| Isobutyl Acetate | Threonine |
| Isobutyl Benzoate | Thuja Occidentalis Leaf Oil |
| Isobutyl Methyl | Thymol |
| Tetrahydropyranol | |
| Isobutyl Palmitate | Thymus Mastichina Oil |
| Isobutyl Pelargonate | Thymus Vulgaris (Thyme) |
| | Extract |
| Isobutyric Acid | Thymus Vulgaris (Thyme) Leaf |
| Isododecane | Thymus Vulgaris (Thyme) Oil |
| Isoeugenol | Thymus Zygis Oil |
| Isoleucine | Tilia Americana Flower Extract |
| Isolongifolene Epoxide | Tilia Cordata Flower Water |
| Isolongifolene Ketone Exo | Tilia Cordata Oil |
| Alpha-Isomethyl Ionone | Tocopherol |
| Isopentanal | Torreya Californica |
| | (California Nutmeg) Oil |
| Isopentylcyclohexanone | Triacetin |
| Isopropyl Acetate | Tribenzoin |
| Isopropyl Alcohol | Tricalcium Phosphate |
| Isopropyl Benzoate | Tricaprin |
| Isopropyl Laurate | Tricaprylin |
| Isopropyl Myristate | Tricyclodecenyl Propionate |
| Isopropyl Palmitate | Tridecyl Alcohol |
| Isopropylphenylbutanal | Triethanolamine |
| Isopulegol | Triethyl Citrate |
| Jasminum Officinale (Jasmine) | Triethylene Glycol |
| Extract | |
| Jasminum Officinale (Jasmine) | Triethylhexanoin |
| Flower Water | |
| Jasminum Officinale (Jasmine) | Trifolium Pratense (Clover) |
| Oil | Flower Extract |
| Juglans Regia (Walnut) Leaf | Trigonella Foenum-Graecum Seed |
| Extract | Extract |
| Juglans Regia (Walnut) Shell | Trimethylamine Oxide |
| Extract | |
| Juniperus Communis Fruit | Trimethylhexanol |
| Extract | |
| Juniperus Communis Fruit Oil | Tromethamine |
| Juniperus Mexicana Oil | Tryptophan |
| Juniperus Oxycedrus Wood Oil | Turpentine |
| Juniperus Oxycedrus Wood Tar | Tyrosine |
| Juniperus Scopulorum Oil | Uikyo Yu |
| Juniperus Virginiana Oil | Gamma-Undecalactone |
| Keihi Ekisu | Undecanoic Acid |
| Keihi Yu | Undecyl Alcohol |
| Ketoglutaric Acid | Undecylenal |
| Kinginka Ekisu | Undecylenic Acid |
| Krameria Triandra Root Extract | Undecylenyl Alcohol |
| Kunzea Ericoides Leaf Oil | Ursolic Acid |
| Lactic Acid | Valeriana Officinalis Root |
| Laminaria Cloustoni Extract | Valine |
| Laminaria Digitata Extract | Vanilla Planifolia Fruit |
| Laminaria Hyperborea Extract | Vanilla Tahitensis Fruit |
| Laminaria Japonica Extract | Vanillin |
| Laminaria Ochroleuca Extract | Vanillyl Butyl Ether |
| Laminaria Saccharina Extract | Verbena Officinalis Extract |
| Lantana Camara Leaf Water | Vetiveria Zizanoides Root Oil |
| Lauraldehyde | Viburnum Prunifolium Extract |
| Lauramine Oxide | Viola Odorata Extract |
| Lauric Acid | Viola Odorata Leaf Extract |
| Laurus Nobilis Leaf | Viola Odorata Leaf Wax |
| Laurus Nobilis Leaf Extract | Viola Odorata Oil |
| Laurus Nobilis Oil | Viola Tricolor Water |
| Lauryl Alcohol | Vitis Vinifera (Grape) Leaf |
| | Oil |
| Lauryl Lactate | Ximenia Americana Seed Oil |
| Lavandula Angustifolia | Xylene |
| (Lavender) Extract | |
| Lavandula Angustifolia | Xylose |
| (Lavender) Flower Extract | |
| Lavandula Angustifolia | Yucca Aloifolia Extract |
| (Lavender) Flower Water | |
| Lavandula Angustifolia | Yucca Filamentosa Extract |
| (Lavender) Oil | |
| Lavandula Hybrida Extract | Yucca Schidigera Extract |
| Lavandula Hybrida Oil | Yucca Vera Extract |
| Lavandula Spica (Lavender) | Yukari Yu |
| Extract | |
| Lavandula Stoechas Extract | Zanthoxylum Acanthopodium |
| | Fruit Oil |
| Lemon Ekisu | Zanthoxylum Americanum Bark |
| | Extract |
| Leptospermum Petersonii Oil | Zanthoxylum Piperitum Oil |
| Leptospermum Scoparium Oil | Zea Mays (Corn) Oil |
| Leucine | Zingiber Officinale (Ginger) |
| | Root Extract |
| Levisticum Officinale Oil | Zingiber Officinale (Ginger) |
| | Root Oil |
| Levulinic Acid | Zingiber Officinale (Ginger) |
| | Water |
| Linalool | |

Desirably, the fragrance blend employed in the instant invention comprises at least three and preferably at least five fragrance components each having peak temperature depression ΔT_{BS} individually of less than 2 and often of up to 1.9. It is especially suitable for the fragrance blend to comprise at least three and particularly at least five fragrance oils, advantageously each having ΔT_{BS} individually of less than 2, for example oils derived from natural products, especially plants, or their synthetic analogues, such oils advantageously appearing in the list hereinabove.

The fragrance (blend) is often incorporated into compositions according to the present invention in a concentration of at least about 0.1% and preferably from 0.5%. Advantageously, by selecting the fragrance in accordance with the present invention, it is possible to employ a higher concentration of the fragrance than for fragrances having a ΔT_{BS} higher than 2. Normally, the fragrance may be incorporated at a concentration of not higher than 4%. of the water-immiscible phase, and in many cases up to 2.5% of the water-immiscible phase. Its concentration is preferably not higher than the concentration at which FF=4,

where FF=[% fragrance in the oil] x ΔT_{BS}, i.e. FF is calculated using the weight fraction of the fragrance in the water immiscible carrier.

In practice, therefore, the FF for the composition is up to 4, and usually at least 0.2. In many practical compositions, FF is at least 1.0. FF is very desirably less than 3 and especially less than 2.0. By employing fragrances with a low standard peak depression ΔT_{BS} it is possible to reduce the risk of gel instability when employing fragrance-sensitive gellants.

Self-evidently, the use of the fragrance according to the instant invention enables the cosmetic producer to incorporate fragrance at concentrations that he has contemplated previously for compositions employing gellants that are less sensitive to fragrances in order to achieve desired perfuming of his compositions. By contrast, many commonly available fragrances have a very high ΔT_{BS}. and could only be incorporated at correspondingly low concentrations before they deleteriously impair the stability of such cosmetic compositions containing fragrance-sensitive gellants, concentrations below desired or optimum perfume levels.

In practice, at the concentrations employed, the fragrance components can dissolve in the water-immiscible carrier liquid or mixture of liquids.

Carrier Liquids

Herein the carrier liquids comprise water-immiscible liquids. Such liquids can be selected from amongst silicone oils, hydrocarbon oils, branched-chain aliphatic alcohols containing at least 12 carbons, aliphatic esters alkylesters of aromatic acids, and polyglycol ethers which have a melting point of below 20° C.

The water-immiscible liquid, that acts as a carrier for a disperse solid or liquid phase, normally comprises one or a mixture of materials which are relatively hydrophobic so as to be immiscible in water. Some hydrophilic liquid may be included in the water-immiscible liquid, to the extent that it is soluble or miscible with the water-immiscible liquid and provided the overall carrier liquid mixture is still immiscible with water. It will generally be desired that this carrier is liquid (in the absence of structurant (gellant)) at temperatures of 15° C. and above. It may have some volatility but its vapour pressure will generally be less than 4 kPa (30 mmHg) at 25° C. so that the material can be referred to as an oil or mixture of oils. More specifically, it is desirable in some embodiments, that at least 80% by weight of the hydrophobic carrier liquid should consist of materials with a vapour pressure not over this value of 4 kPa at 25° C.

It is preferred, e.g. for use in cosmetic formulations that the hydrophobic carrier material includes a volatile liquid silicone, i.e. liquid polyorganosiloxane. To class as “volatile” such material should have a measurable vapour pressure at 20 or 25° C. Typically the vapour pressure of a volatile silicone lies in a range from 1 or 10 Pa to 2 kPa at 25° C.

It is desirable to include volatile silicone because it gives a “drier” feel to the applied film after the composition is applied to skin.

Volatile polyorganosiloxanes can be linear or cyclic or mixtures thereof. Preferred cyclic siloxanes include polydimethylsiloxanes (polydimethicones) and particularly those containing from 3 to 9 silicon atoms and preferably not more than 7 silicon atoms and most preferably from 4 to 6 silicon atoms, otherwise often referred to as cyclomethicones. Preferred linear siloxanes include polydimethylsiloxanes containing from 3 to 9 silicon atoms.

The volatile siloxanes normally by themselves exhibit viscosities of below 10⁻⁵ m²/sec (10 centistokes), and particularly above 10⁻⁷ m²/sec (0.1 centistokes), the linear siloxanes normally exhibiting a viscosity of below 5×10⁻⁶ m²/sec (5 centistokes). The volatile silicones can also comprise branched linear or cyclic siloxanes such as the aforementioned linear or cyclic siloxanes substituted by one or more pendant —O—Si(CH₃)₃ groups. Examples of commercially available silicone oils include oils having grade designations 344, 345, 244, 245 and 246 from Dow Corning Corporation; Silicone 7207 and Silicone 7158 from Union Carbide Corporation; and SF1202 from General Electric.

The hydrophobic water-immiscible liquid carrier employed in many compositions herein can alternatively or additionally comprise non-volatile silicone oils, which include polyalkyl siloxanes, polyalkylaryl siloxanes and polyethersiloxane copolymers. These can suitably be selected from dimethicone and dimethicone copolyols. Selected polyalkylaryl siloxanes include short chain polysiloxanes, e.g. tri or tetrasiloxanes containing on average at least one phenyl group per siloxane unit, for example tetraphenyltrisiloxanes. Commercially available non-volatile silicone oils include Dow Corning 556, Dow Corning 200 series and DC704.

The water-immiscible liquid carrier may contain from 0 to 100% by weight of one or more liquid silicones. Some embodiments contain liquid silicones in at least 10%, better at least 15%, by weight of the whole composition. If silicone oil is used, in some embodiments, volatile silicone preferably constitutes from 10 to 100% of the weight of the carrier liquid. In many instances, when a non-volatile silicone oil is present, its weight ratio to volatile silicone oil is chosen in the range of less than 3:1 such as from 1:3 to 1:40, whereas in certain other embodiments, the proportion of volatile silicone oils is from 0 to less than 10%, so that the weight ratio of non-volatile to volatile silicone oils is greater than 10:1, such as from 15:1 to ∞:1. In other embodiments, liquid silicones are absent, or present in only a small proportion of the water-immiscible phase, such as up to 7 or 8% by weight. Accordingly, a range of mixtures of silicone oils and non-silicone oils can be employed as liquid carrier for structuring by the CHME invention esters. Many of such mixture employ a weight ratio of the silicone to non-silicone oils of from 4:1 to 1:4. The selection of carrier fluids is often made taking into account the refractive index of the components of the carrier fluid mixture, and the refractive index of a particulate active constituent such as an antiperspirant or of a water-miscible phase.

Silicon-free hydrophobic liquids can be used instead of, or in some embodiments in addition to liquid silicones. Silicon-free hydrophobic organic liquids which can be incorporated include volatile or non-volatile liquid aliphatic hydrocarbons such as mineral oils or hydrogenated polyisobutene, often selected to exhibit a low viscosity. Further examples of liquid hydrocarbons are polydecene and paraffins and isoparaffins of at least 10 carbon atoms.

Other hydrophobic carriers are liquid aliphatic or aromatic esters, but for some uses, for example antiperspirant formulations, and especially in formulations gelled with an acylated sugar, these should be used as only part of the liquid carrier, desirably not above 20%, and possibly less than 10% and advantageously less than 5% by weight of the water-immiscible liquid carrier, less than including 0%, i.e. in advantageous formulations the liquid ester carrier is absent. The total weight of such esters in the liquid carrier desirably includes any such esters which may be introduced via the fragrance blend.

Suitable aliphatic esters contain at least one long chain alkyl group, such as esters derived from C₁ to C₂₀ alkanols esterified with a C₈ to C₂₂ alkanoic acid or C₆ to C₁₀ alkanedioic acid. The alkanol and acid moieties or mixtures thereof are preferably selected such that they each have a melting point of below 20° C. These esters include isopropyl myristate, lauryl myristate, isopropyl palmitate, diisopropyl sebacate and diisopropyl adipate.

Suitable liquid aromatic esters, preferably having a melting point of below 20° C., include fatty alkyl benzoates. Examples of such esters include suitable C₈ to C₁₈ alkyl benzoates or mixtures thereof.

Further instances of suitable hydrophobic carriers comprise liquid aliphatic ethers derived from at least one fatty alcohol, such as myristyl ether derivatives e.g. PPG-3 myristyl ether or lower alkyl ethers of polygylcols, eg C2-C₄ alkyl PPG ethers such as commercial products having CFTA nominally labelled PPG-14 butyl ether.

Aliphatic alcohols which are solid at 20° C., such as stearyl alcohol are preferably absent or present in low concentration such as less than 5% by weight of the whole composition since these lead to visible white deposits when a composition is used.

However, aliphatic alcohols which are liquid at 20° C. may be employed. These include branched chain alcohols of at least 10 carbon atoms such as isostearyl alcohol and octyl dodecanol.

Silicon-free liquids can constitute from 0-100% of the water-immiscible liquid carrier. It is preferred that silicone oil and/or a hydrocarbon oil is present and that the total amount of other liquid carriers, preferably, constitutes up to 50 or 60% for example from 0 to 10% or 10 to 20% by weight of the water-immiscible carrier liquid.

An especially desired combination of water immiscible carrier liquids comprises a mixture of a silicone liquid such as a cyclomethicone and a hydrocarbon liquid, such as in a weight ratio of the former to the latter of from 3:2 to 1:10, optionally in the presence of an emollient water-immiscible liquid.

The proportion of the water-immiscible carrier liquids in the cosmetic composition is often selected in the range of from 15 to 95% by weight, and in many embodiments is from 30 to 80% by weight. Where the water-immiscible carrier constitutes the sole liquid phase, its weight proportion is conveniently in the range of at least 50% and/or in desirable practice up to 75%.

A number of compositions according to the present invention are in the form of an emulsion, having a disperse hydrophilic phase, which is to say aqueous or water-miscible. The hydrophilic disperse phase in such an emulsion normally comprises water as solvent and can comprise alternatively or additionally one or more water-soluble or water-miscible liquids. The proportion of hydrophilic carrier fluid, e.g. water, in the disperse phase, in an emulsion according to the present invention is often selected in the range of up to 60%, and particularly from 10% up to 40% or 50% of the whole formulation.

One class of water-soluble or water-miscible liquids comprises short chain monohydric alcohols, e.g. C₁ to C₄ and especially ethanol or isopropanol, which can impart a deodorising capability to the formulation. A further class of hydrophilic liquids comprises diols or polyols preferably having a melting point of below 40° C., or which are water miscible. Examples of water-soluble or water-miscible liquids with at least one free hydroxyl group include ethylene glycol, 1,2-propylene glycol, 1,3-butylene glycol, hexylene glycol, diethylene glycol, dipropylene glycol, 2-ethoxyethanol, diethylene glycol monomethylether, triethyleneglycol monomethylether and sorbitol. Especially preferred are propylene glycol and glycerol.

In an emulsion, the disperse phase is likely to constitute from 5 to 80 or 85% of the weight of the composition preferably from 5 to 50 or 65%, more preferably from −25 or 35% up to 50 or 65%, while the continuous phase with the structurant therein provides the balance from 15 or 35% up to 95% of the weight of the composition. Advantages can accrue when the internal phase weight constitutes a minor proportion of emulsion, such as from about 30 to 45% by weight. Yet other advantages arise at 45 to 65% internal phase weight.

An emulsion composition will generally include one or more emulsifying surfactants which may be anionic, cationic, zwitterionic and/or nonionic surfactants. Nonionic surfactants are preferred. The proportion of emulsifier in the composition is often selected in the range up to 10% by weight and in many instances from 0.1 up to 5% by weight of the composition. Most preferred is an amount from 0.1 up to 1% by weight, such as 0.3%, 0.4% or 0.5% by weight.

Nonionic emulsifiers are frequently classified by HLB value (hydrophylic/lipophilic balance). It is desirable to use an emulsifier or a mixture of emulsifiers with an overall HLB value in a range from 2 to 10 preferably from 3 to 8.

The classes of emulsifiers for making a water-in-oil emulsion well known to the skilled man can be employed herein. Such emulsifiers include polyethoxylated and/or polypropoxylated fatty alcohols or fatty acids having a suitable HLB value. Other suitable emulsifiers include dimethicone copolyols in which a polysiloxane is include polyethoxylated and/or polypropoxylated.

Gellants for the Carrier

The fragrance-sensitive gellants for the water-immiscible carrier liquids herein normally are found within the class of fibre forming gellants. Whether or not the gellant is fragrance-sensitive can be determined by conducting the stability test described hereinbefore. Fibre-forming gellants are non-polymeric substances that on solidification in a water-immiscible carrier liquid form long fibres or have a diameter of not greater than 1 μm and an aspect ratio of at least 10:1. Aspect ratio herein indicates the ratio of fibre length to fibre diameter. In many fibre-forming gellants, the fibre diameter is less than 0.5 μm and in some is less than 0.2μ.

Materials with this property (fibre-forming gellants) have been reviewed by Terech and Weiss in “Low Molecular Mass Gelators of Organic Liquids and the Properties of their Gels” Chem. Rev 97, 3133-3159 [1997] and by Terech in Chapter 8, “Low-molecular weight Organogelators” of the book “Specialist surfactants” edited by I D Robb, Blackie Academic Professional, 1997.

The conditions under which cosmetic compositions are destabilised by the incorporation of fragrances and/or the extent of destabilisation tend to vary in accordance with the sub-class of fibre-forming gellant.

Preferably, the instant invention is employed for cosmetic compositions in which the fibre-forming gellants comprise one or more ester groups or a sterol. Compositions containing such gellants are particularly susceptible to being destabilised by incorporating fragrances.

A sub-class of fibre forming gellants for which the present invention is particular suitable comprises sugar esters, which contain many ester groups, including in particular esters of cellobiose or maltose. Preferably, the proportion of esterified hydroxyl groups in the sugars is at least 75% preferably at least 87.5% and particularly at least 95%. In a number of preferred esterified sugars, they are esterified at their anomeric carbon to an extent of at least 80%. Preferably, the sugar esters are esters of intermediate chain aliphatic fatty acids, the acids desirably containing from 7 to 12 carbons, particularly containing 9 or 10 carbons and especially containing 9 carbon atoms. Linear acyl groups are especially desired. In many embodiments, all the acyl substituents nominally contain the same number of carbons, most desirably 9, i.e. any variation being by way of impurity. An especially desirable fibre-forming gellant for use in the instant invention is called cellobiose octanonanoate. Such a material, by virtue of its manufacture, commonly comprises a small fraction of cellobiose heptanonanoate, such that the average extent of esterification is from 95 to 99.5%. Cellobiose octanonanoate desirably is at least 75% and in various highly desired embodiments is at least 80% in the α anomer, the residue being in the β anomer. It is an advantage of the present invention that it eases the fragrancing of cosmetic compositions employing such ester gellants.

Although it is convenient for all the acyl substituents to be the same, subject as ever to the presence of impurity proportions of related acyl groups present in the acid feedstock, in some embodiments, it can be desirable for a second and different acyl substituent to be present, and particularly for different substitution to occur at the anomeric carbon of the sugar. In such an double esterified sugar, it is particularly esterified by the second acyl substituent to the extent of from 5 to 12.5%. That corresponds to from 40 to 100% esterification solely at the anomeric carbon of the sugar.

The second acyl substituent can be derived from aliphatic acids, cycloaliphatic acids or aromatic acids. The aliphatic acids can contain from 4 to 24 carbon atoms, but of course contain a different number of carbon atoms from that in the principal acyl substituent in order to be different. Preferably, the number of carbons in the second acyl group, if aliphatic, is either at least 2 more than or at least 2 less than the number of carbons in the principal acyl substituent. If the principal acyl substituent contains 9 carbons, then the second aliphatic acyl group preferably contains up to 7 or at least 11 carbons.

Where the second acyl substituent comprises a cycloaliphatic group, one suitable example is the acyl residue of cyclohexylcarboxylic acid, and where it comprises an aromatic group, it is for example the residue from an aryl acid such as benzoic or napthoic acid. By substituting a second and different acyl group in the sugar, it is possible to alter the gelation properties of the gellant.

A second class of fibre-forming gellants for which this invention is particular suitable comprises sterols. Within that class, lanosterol is particular example meriting mention.

The weight proportion of gellant in the cosmetic composition to employ is usually selected in the range of from at least 0.5 and in many compositions at least 1.5%. The weight proportion of such gellants is normally not greater than 20% and commonly is not more than 15%. The actual amount selected for a particular fibre-forming gellant will, as is conventional, take into account the carrier liquids in which the cosmetic active is suspended, its capability to gel such carrier liquids, the extent of hardness of the gel desired and conditions in which the composition will be employed or stored.

Cosmetic Active Materials

Although the present invention is suitable for cosmetic compositions in general, it is particular suitable for compositions which contain an astringent antiperspirant salt, preferably in an amount of from 0.5-60%, particularly from 5 to 30% or 40% and especially from 5 or 10% to 30 or 35% of the weight of the composition.

The astringent antiperspirant actives for use herein are preferably selected aluminium, zirconium and mixed aluminium/zirconium salts, including both inorganic salts, salts with organic anions and complexes. Preferred astringent salts include aluminium, zirconium and aluminium/zirconium halides and, especially halohydrate salts, such as chlorohydrates.

Aluminium halohydrates are usually defined by the general formula Al₂(OH)ₓQ_{y}._{w}H₂O in which Q represents chlorine, bromine or iodine, x is variable from 2 to 5 and x+y=6 while wH₂O represents a variable amount of hydration. Especially effective aluminium halohydrate salts, known as activated aluminium chlorohydrates, are described in EP-A-6739 (Unilever NV et al), the contents of which specification is incorporated herein by reference. Some activated salts do not retain their enhanced activity in the presence of water but are useful in substantially anhydrous formulations, i.e. formulations which do not contain a distinct aqueous phase.

Zirconium actives can usually be represented by the empirical general formula: ZrO(OH)_{2n-nz}B_{z.}wH₂O in which z is a variable in the range of from 0.9 to 2.0 so that the value 2n-nz is zero or positive, n is the valency of B, and B is selected from the group consisting of chloride, other halide, sulphamate, sulphate and mixtures thereof. Possible hydration to a variable extent is represented by wH2O.

Preferable is that B represents chloride and the variable z lies in the range from 1.5 to 1.87. In practice, such zirconium salts are usually not employed by themselves, but as a component of a combined aluminium and zirconium-based antiperspirant.

The above aluminium and zirconium salts may have coordinated and/or bound water in various quantities and/or may be present as polymeric species, mixtures or complexes. In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and P-alanine, and preferably glycine which has the formula CH₃CH(NH₂)CO₂H.

It is highly desirable to employ complexes of a combination of aluminium halohydrates and zirconium chlorohydrates together with amino acids such as glycine, which are disclosed in U.S. Pat. No. 3,792,068 (Luedders et al). Certain of those Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine. Actives of this preferred type are available from Westwood, from Summit and from Reheis.

Other actives that may be utilised include astringent titanium salts, for example those described in GB 2299506A.

A number of preferred formulations comprise from 15 to 26% by weight antiperspirant active, 50 to 79.5% carrier liquid, 5 to 15% fibre-forming gellant and 0.5 to 2% fragrance having ΔT_{B} of not more than 2, advantageously the antiperspirant actives, carrier liquid, gellant and fragrance being selected in accordance with the descriptions of such materials identified herein above.

Other preferred formulations comprise from 40 to 70% by weight of a continuous water-immiscible phase containing from 30 to 65% of a water-immiscible carrier liquid, 5 to 15% fibre-forming gellant and 0.5 to 2% fragrance having ΔT_{B} of not more than 2, and from 30 to 60% by weight of a disperse hydrophilic phase containing from 15 to 26% by weight antiperspirant active, and from 0.15 to 2% by weight of an emulsifier, advantageously the antiperspirant actives, carrier liquids, gellant fragrance and emulsifier being selected in accordance with the descriptions of such materials identified herein above.

Translucent/Transparent Compositions

In some highly desirable embodiments herein, the compositions are translucent/transparent. For an emulsion, this can be achieved by matching the refractive indices of the water-immiscible continuous phase and the polar or aqueous disperse phase, the value of refractive index at which they are matched also approximately matching the refractive index of the structurant.

The refractive index of a fibre-forming structurants contemplated herein can be determined by using that structurant to gel a number of oils or oil mixtures of differing refractive index and in many instances are in the range of from 1.43 to 1.5.

For the continuous phase, silicon-free water-immiscible liquid oils described hereinbefore generally have refractive indices in a range from 1.43 to 1.49 at 22° C. and can be used alone or mixed together to give a silicon-free carrier liquid with refractive index in this range. Volatile silicone oils generally have a refractive index slightly below 1.40 at 2° C. and some non-volatile silicone oils, eg dimethicone oils, similarly have a refractive index of about 1.41 at 22° C., but carrier liquid mixtures with refractive indices in the range from 1.41 to 1.46 can be obtained by mixing volatile or such non-volatile silicone with other oils. Other non-volatile silicone oils containing aryl substitution generally have refractive indices of at least 1.45, for example from 1.45 to 1.48 at 22° C., the oils bearing a high ratio of phenyl substituents to alkyl substituents can enjoy a higher refractive index than 1.48, such as from 1.49 to 1.56. Such other aforementioned non-volatile silicone oils can be included when desired to achieve a carrier liquid mixture having a desired refractive index.

The RI of the structured continuous phase will conveniently be very close to the RI of the carrier liquid (usually a carrier liquid mixture) which is its principal component.

For the disperse phase, a solution of an antiperspirant active salt in water alone will generally display a refractive index below 1.425. The refractive index can be raised by incorporating a diol or polyol into the aqueous solution. It is believed to be beneficial to match the refractive index of a polar disperse phase to that of a structurant network within a continuous phase. Moreover, it can be achieved without using so much diol or polyol as will make the composition excessively sticky.

Optional Constituents

Optional ingredients include wash-off agents, often present in an amount of up to 10% w/w to assist in the removal of the formulation from skin or clothing. Such wash-off agents are typically nonionic surfactants such as esters or ethers containing a C₈ to C₂₂ alkyl moiety and a hydrophilic moiety which can comprise a polyoxyalkylene group (PbE or POP) and/or a polyol.

The compositions herein can incorporate one or more cosmetic adjuncts conventionally contemplatable for cosmetic solids or soft solids. Such cosmetic adjuncts can include skin feel improvers, such as talc or finely divided polyethylene, for example in an amount of up to about 10%; skin benefit agents such as allantoin or lipids, for example in an amount of up to 5%; colours; skin cooling agents other than the already mentioned alcohols, such a menthol and menthol derivatives, often in an amount of up to 2%, all of these percentages being by weight of the composition. Refractive Indexes of constituents or phases herein and refractive index matching is conducted at 22° C. herein, unless otherwise specified.

Preparation of Invention Compositions

Compositions according to the present invention can be made in accordance with known methods for making sticks in which a water-immiscible carrier liquid is gelled with the respective class of gellant such as a fibre-forming gellant.

In particular, the invention compositions can be made by dissolving the gellant, eg the fibre-forming gellant in the water-immiscible carrier liquid at an elevated temperature, commonly in the range of at least 60° C. and often from 75 to 95° C., mixing the resultant solution with a cosmetic adjunct, such as an antiperspirant salt and with the fragrance, introducing the resultant mixture whilst still mobile into a dispenser for the composition or a mould and thereafter cooling the mixture to below its solidification temperature, which is often in the range of from 45 to 55° C. Preferably, introduction of the fragrance into the mixture is delayed until the mixture is from 5 to 15° C. above its solidification temperature so as to minimise any subsequent degradation of the fragrance, such as loss of volatile components, before the mixture solidifies.

When the composition is in the form of an emulsion, the foregoing process is commonly modified by forming two phases which are mixed together before being charged into a container or mould. One of the phases is hydrophilic and comprises a mixture of water and water-miscible and/or water-soluble ingredients such as in particular the antiperspirant active salt, and any di or trihydric alcohol. If necessary the hydrophilic phase can be heated to an elevated temperature such as from 70 to 90° C. in order to accelerate phase homogenisation and/or salt dissolution. The hydrophobic phase can be made in accordance with the process given above for anhydrous compositions. The emulsifier can be mixed with either phase at the discretion of the producer, but preferably in that with which it is more compatible, chosen on the basis of its HLB value. The two phases are then homogenised together and preferably allowed to cool to less than 10° C. above the stick solidification temperature before the fragrance is introduced.

Product Dispenser

Emulsion sticks according to the present invention are normally housed in dispensing containers, the shape and size of which, the materials of their construction and the mechanisms employed therein for dispensing the sticks are those commensurate with the chosen cosmetic. Thus, by way of example, an antiperspirant or deodorant stick is often housed in a barrel, commonly of circular or elliptical transverse cross section, having an open end through which the stick can pass and an opposed closed end, commonly comprising a platform or elevator that is axially moveable along the barrel. The platform can be raised by the insertion of a finger or more commonly by rotation of an externally exposed rotor wheel that rotates a threaded spindle extending axially through a co-operating threaded bore in the platform. The barrel normally also has a removable cap that can fit over its open end. The barrel is normally made from an extrudable thermoplastic such as polypropylene or polyethylene.

The present invention also provides cosmetic products comprising an invention cosmetic stick as described hereinbefore disposed within a dispensing barrel.

Determination of Product Hardness

The hardness of the cosmetic compositions can be determined by a conventional method. One such method employs a sphere which is dropped onto an exposed flat surface of the composition and a second such method employs a needle which penetrates into the composition, the depth of the indentation or the penetration varying inversely with the hardness of the composition. Hardness tests are conducted at laboratory ambient, normally 22° C.

Conventional Sphere Indentation Method

This test apparatus can move a blunt probe, a sphere, into or out from a sample at a controlled speed and at the same time measure the applied force. The parameter which is determined as hardness is a function of the peak force and the projected area of indentation.

A specific conventional test protocol uses a Stable Micro systems TA.XT2i Texture Analyser. A metal sphere, of diameter 9.5 mm, is attached to the underside of the Texture Analyserσ 5 kg load cell such that it could be used for indenting a sample placed beneath it on the base plate of the instrument. After positioning the sample, the sphere position is adjusted until it is just above the sample surface. Texture Expert Exceed software can be used to generate the subsequent motion profile used in the test method. This profile initially indented the sphere into the sample at an indentation speed of 0.05 mm/s until a designated force is reached, chosen such that the distance of penetration into the sample is less than the radius of the sphere. At this load, the direction of motion of the sphere is immediately reversed to withdraw the sphere from the sample at the same speed of 0.05 mm/s. During the course of the test, the data acquired are time(s), distance (mm) and force (N) and the data acquisition rate is 25 Hz.

Suitable samples for measurement are contained in stick barrels, which had a screw mechanism, if firm solids, at least on manufacture. The stick is wound up until it protrudes above the top edge of the barrel and then a knife is used to skim the top of the barrel in such a way as to leave a flat uniform surface. The stick is then pushed back into the barrel as far as possible to minimise any mechanical interference resulting from the compliance of the central screw mechanism in the pack. Two indents are generally made either side of the screw.

The various items of data associated with each test are manipulated using standard spreadsheet software and used to calculate the hardness, H, in the following equation:

$$H [ N / {mm}^{2} ] = \frac{{F}_{max} [ N ]}{{A}_{p} [ {mm}^{2} ]}$$

where Fₘₐₓ is the peak load and Aₚ is the projected area of the indentation remaining on unloading. This area can be calculated geometrically from the plastic indentation depth. This is slightly less than the total penetration depth measured under load because of elastic deformation of the sample. The plastic indentation depth is calculated from a graph of the unloading-force-versus-total-penetration-depth. The initial slope of this unloading data depends on the initial elastic recovery of the sample. The plastic indentation depth is estimated from an intercept between the zero force axis and a straight line drawn at a tangent to the initial part of the unloading slope.

Similar hardness measurements can also be carried out using a desktop Instron Universal Testing Machine (Model 5566) fitted with a 10 N load cell, the data analysis being performed in the same way.

For a solid cosmetic composition the measured hardness will generally be greater than 0.5 Newton/mm², and preferably greater than 1 Newton/mm².

Conventional Needle Penetrometer Method

The hardness and rigidity of a composition which is a firm solid can be determined by penetrometry. If the composition is a softer solid, this will be observed as a reduced or lack of any resistance to the penetrometer probe.

A suitable procedure is to utilises a lab plant PNT penetrometer equipped with a Seta wax needle (weight 2.5 grams) which has a cone angle at the point of the needle specified to be 9° 10′+/−15″. A sample of the composition with a flat upper surface is used. The needle is lowered onto the surface of the composition and then a penetration hardness measurement is conducted by allowing the needle with its holder to drop under a total weight, (i.e. the combined weight of needle and holder) of 50 grams for a period of five seconds after which the depth of penetration is noted. Desirably the test is carried out at a number of points on each sample and the results are averaged. Utilising a test of this nature, an appropriate hardness for use in an open-ended dispensing container is a penetration of less than 30 mm in this test, for example in a range from 2 to 30 mm. A penetration of greater than 30 mm indicates a soft solid. Preferably the penetration is in a range from 5 mm to 20 mm for a firm stick.

In determining whether or not the gellant is fragrance sensitive, the tester will employ the same apparatus and test conditions to test the stick both before and after storage.

Having given a detailed description of the invention and preferred embodiments thereof, further compositions will now be described by way of example only.

In the following Examples and Comparisons, the TBS values for reference fragrance-free compositions and compositions containing the fragrance or its individual components were measured in the following procedure using a Perkin Elmer Pyris 1 differential scanning calorimeter. Likewise, the same procedure was employed to measure FF values directly employing a concentration of the fragrance different from 1%.

Samples of approximately 20 mg, typically between 15 mg and 25 mg, of the composition to be tested were enclosed in hermetically sealed stainless steel pans. An empty pan was used as the physical reference. The samples and the empty pan were then subjected to the following temperature programme:
(Step 1) Hold at 25° C. for 5 minutes.
(Step 2) Heat at 10 K/min to 70° C.
(Step 3) Hold at 70° C. for 30 seconds.
(Step 4) Cool at 10 K/min to −20° C.
(Step 5) Hold at −20° C. for 1 minute.
(Step 6) Heat at 10 K/min to 70° C.

Data collected from the empty pan was subtracted from data for each sample and any remaining slope in the baseline cancelled as is common practice in differential scanning calorimetry. From the graphical plot of heat flow against temperature, the temperature was identified at which the second heat flow maximum occurs. This was commonly between 40° C. and 50° C. for unfragranced samples T_{BN}, and between 35° C. and 50° C., for fragranced samples T_{BF}(T_{BFS} when measured under standard conditions).

ΔT_{B} for the fragranced composition was calculated from the following equation.
ΔT_{B}=T_{BN}−T_{BF}— and
under standard conditions the standard peak depression for the fragrance is given by
ΔT_{BS}=T_{BN}−T_{BFS}.

Where ΔT_{B} was measured for compositions containing other than at 1 wt % fragrance (i.e. under non-standard conditions) or for fragrance components without measuring the standard ΔT_{B}S for a blend of components, an approximate ΔT_{BS} was calculated by assuming that ΔT_{B} varied proportionately to its concentration or by weight averaging the respective ΔT_{BS} for the individual components or sub-combinations of components. ΔT_{BS} for those minor other ingredients designated by * is an assumed value.

The reference formulation in which ΔT_{BS} for fragrances was measured is summarised in Table 1 below in which the weight ratio of hydrogenated polydecene to volatile silicones was 60:40.

TABLE 1

| | |
|---|---|
| | % by |
| Constituent | weight |

Disperse phase

| | | | |
|---|---|---|---|
| antiperspirant | Aluminium | Summit Q5 7167 ™ | 24.87 |
| active - | zirconium | | |
| | glycine complex | | |
| water | | | 20.07 |
| humectant | glycerol | | 4.82 |
| | emulsifier | Abil EM90 ™ | 0.49 |

Continuous phase

| | | | |
|---|---|---|---|
| carrier liquid | cyclomethicone | DC 245 ™ | 15.52 |
| C1 | | | |
| carrier liquid | Hydrogenated | Silkflo 364NF ™ | 23.28 |
| C2 | Polydecene | | |
| gellant G1 | cellobiose | | 9.95 |
| | octanonanoate | | |
| Fragrance | various | | 1.0 |

The fragrances and their components employed in Examples 1 to 7 and Comparisons A to E are summarised in Table 2 below, 5 as well as the time before the sample was considered to lack acceptable stability.

The samples for determining composition stability were made in a standard method for making an emulsion stick containing an antiperspirant active in which the water-immiscible continuous phase was structured with gellant G1 cellobiose octanonanoate (>95% esterified, >90% a anomer, made in accordance with the description in EP1199311), each sample containing 1% by weight of the selected fragrance. The aqueous phase materials, viz water, glycerol and antiperspirant active were mixed and heated to 80° C. until complete solids dissolution had occurred and allowed to cool to 70° C. The carrier liquids, gellant and emulsifier were mixed together in a Silverson™ mixer at 2500 rpm and heated up to 95° C. to ensure dissolution of the gellant, and then allowed to cool to 85° C. The aqueous mixture was then poured slowly into the carrier liquid mixture and the speed of mixing increased to 7500 rpm for 5 minutes, during which time the sample cooled to about 77°. The mixture was then poured into small clear glass vials which contained the weighted amount of selected fragrance, and stirred with a clean spatula. The vials were then tightly sealed and the following morning were transferred to an oven maintained at 45° C. in which they were subsequently stored.

The samples were inspected periodically to see whether their appearance had changed, indicating that they were no longer stable. Inspections took place after 1 day, 7 days, 13 days, 20 days, 27 days, and 41 days or after 1 day, 7 days, 14 days, 21 days, 28 days, 42 days and 56 days. The sample was considered no longer to be stable if it exhibited crystallisation, mottling or presence of a melted fraction.

TABLE 2

| INGREDIENT | ΔT_{BS} | Ex1 | Ex2 | Ex3 | Ex4 | Ex5 | Ex6 | Ex7 | CA | CB | CC | CD | CE |
|---|---|---|---|---|---|---|---|---|---|---|---|---|---|
| Allyl amyl glycolate | 1.463 | | 20 | 20 | | | | | | | | | |
| Alpha ionone | 1.873 | | 20 | | | | | | | | 20 | | |
| Other ingredient #1 | 1.254 | | | | 6 | 5 | | | | | | | |
| Other ingredient #2 | | | | | | | | | | | | | |
| (10% in DPG) | | | | | | | | | | | | | |
| equivalent to . . . | | | | | | | | | | | | | |
| Other ingredient #2 | 0.932 | | | | | | | 0.13 | | | | | |
| DPG | 0.176 | | | | | | | 1.17 | | | | | |
| Benzyl acetate | 2.231 | | | | 8 | 5 | | | 20 | | 20 | | 20 |
| Benzyl salicylate | 1.963 | | | | | | | 5 | | 20 | | | 20 |
| Bourgeonal TM | 2.648 | | | | | | 1 | | | 20 | | 20 | |
| (Quest) | | | | | | | | | | | | | |
| Other ingredient #3 | 1.600 | | | | | 2.5 | | | | | | | |
| Other ingredient #4 | | | | | | | | | | | | | |
| (10% in DPG) | | | | | | | | | | | | | |
| equivalent to . . . | | | | | | | | | | | | | |
| Other ingredient #4* | 1.900 | | | | | | | 0.1 | | | | | |
| DPG | 0.176 | | | | | | | 0.9 | | | | | |
| Other ingredient #5 | 1.563 | | | | | | | 4 | | | | | |
| Other ingredient #6* | 1.900 | | | | | | 7 | | | | | | |
| Other ingredient #7 | | | | | | | | | | | | | |
| (10% in DPG) | | | | | | | | | | | | | |
| equivalent to . . . | | | | | | | | | | | | | |
| Other ingredient #7* | 1.900 | | | | | | | 0.1 | | | | | |
| DPG | 0.176 | | | | | | | 0.9 | | | | | |
| Diethyl phthalate | 1.974 | | 20 | | 20 | | | | | | | | |
| Dihydromyrcenol | 2.367 | | | | | | | | | | 20 | | 20 |
| Other ingredient #8* | 1.900 | | | | | | 1 | | | | | | |
| Dipropylene glycol (DPG) | 0.176 | | | | | | 30 | | | | | | |
| Ethylene brassylate | 1.863 | | 20 | | | | | 7 | 20 | | | | |
| Other ingredient #9 | 2.499 | | | | 3 | | | 0.5 | | | | | |
| Other ingredient #10 | | | | | | | | | | | | | |
| (10% in DEP) | | | | | | | | | | | | | |
| equivalent to . . . | | | | | | | | | | | | | |
| Other ingredient #10* | 1.900 | | | | 0.4 | | | | | | | | |
| DEP | 1.974 | | | | 3.6 | | | | | | | | |
| Hexyl cinnamic | 1.670 | | | 20 | | | | 4 | | | | | |
| aldehyde | | | | | | | | | | | | | |
| Other ingredient #11 | 1.763 | | | | 5 | | | 7 | | | | | |
| Iso ambois TM | 0.964 | 20 | | | 6 | 10 | | | | | | | |
| (Quest) | | | | | | | | | | | | | |
| Isobornyl acetate | 1.258 | 20 | | 20 | | | | | | | | | |
| Jasmopyrane TM | 1.241 | 20 | | | | | | | 20 | | | | |
| (Quest) | | | | | | | | | | | | | |
| Other ingredient #12 | | | | | | | | | | | | | |
| (10% in DEP) | | | | | | | | | | | | | |
| equivalent to . . . | | | | | | | | | | | | | |
| Other ingredient #12* | 1.900 | | | | | 0.06 | | | | | | | |
| DEP | 1.974 | | | | | 0.54 | | | | | | | |
| Other ingredient #13* | 1.900 | | | | | | 6 | | | | | | |
| (4-(1- | 3.178 | | | | | | | | 20 | | 20 | 20 | 20 |
| Methylethyl)cyclohexyl) | | | | | | | | | | | | | |
| methanol | | | | | | | | | | | | | |
| Methyl | 2.064 | | 20 | | 8 | 12.5 | 16 | 20 | | | | | |
| dihydrojasmonate | | | | | | | | | | | | | |
| Other ingredient #14* | 1.900 | | | | | | | 0.3 | | | | | |
| Other ingredient #15* | 1.900 | | | | 10 | | | 6.9 | | | | | |
| 1-Oxa-5(6)- | 0.864 | 20 | | 20 | 10 | | | 7 | | | | | |
| cyclohexadecen- | | | | | | | | | | | | | |
| 16-one | | | | | | | | | | | | | |
| Other ingredient #16* | 1.900 | | | | 10 | | 11 | | | | | | |
| 2-phenylethanol | 4.581 | | | | | | 10 | | 20 | 20 | 20 | 20 | 20 |
| Other ingredient #17* | 1.900 | | | | | | 5 | | | | | | |
| Other ingredient #18* | 1.900 | | | | 10 | 9.4 | | 6.8 | | | | | |
| Rossitol TM | 2.283 | | | | | | | 1 | | 20 | | 20 | |
| (Quest) | | | | | | | | | | | | | |
| Other ingredient #19* | 1.900 | | | | | 2.5 | | | | | | | |
| Other ingredient #20* | 1.900 | | | | | 2.5 | | | | | | | |
| Tetrahydrolinalol | 1.671 | | | | | | | 4 | | | | | |
| Other ingredient #21* | 1.900 | | | | | | 3 | | | | | | |
| Triethyl citrate | 2.804 | | | | | 25 | | 15 | | | | 20 | |
| Other ingredient #22 | | | | | | | | | | | | | |
| (10% in DPG) | | | | | | | | | | | | | |
| equivalent to . . . | | | | | | | | | | | | | |
| Other ingredient #22* | 1.900 | | | | | | | 0.12 | | | | | |
| DPG | 0.176 | | | | | | | 1.08 | | | | | |
| TOTAL of blend (%) | | 100 | 100 | 100 | 100 | 100 | 100 | 100 | 100 | 100 | 100 | 100 | 100 |
| Fraction of blend with | | 100 | 100 | 100 | 69.6 | 85.5 | 67.0 | 85.7 | 100 | 100 | 100 | 100 | 100 |
| B measured (%) | | | | | | | | | | | | | |
| ΔT_{BS} of the | | 1.13 | 1.85 | 1.32 | 1.77 | 1.89 | 1.64 | 1.85 | 2.62 | 2.56 | 2.85 | 3.10 | 2.86 |
| fragrance blend | | | | | | | | | | | | | |
| Days to Instability for | | 27 to | 27 to | 27 to | 28 to | 42 to | 28 to | 28 to | <7 | <7 | 1 to −7 | 1 | 1 |
| Duplicated Samples | | 41 | 41 | 41 | 56 | 56 | 42 | 42 | | | | | |

From Table 2, it can be seen that compositions containing those fragrances which have a ΔT_{BS} of less than 2 achieved significantly longer stability than did corresponding compositions containing fragrances with a ΔT_{B} of greater than 2. None of the compositions in Comparisons CA to CE achieved a stability of greater than 7 days, and even that figure is of doubtful provenance since it is 7 times greater than the previously length of elapsed storage time. On the other hand, the least period of observed-stability for Examples 1 to 7 was 27 or 28 days and in several instances was 2 or 4 weeks longer.

Secondly, it can be seen from Table 2 such as from Example 5 and 7 that it was possible to include a significant, though minor, fraction of a fragrance component having a ΔT_{BS} of greater than 2 and still achieve composition stability provided that the fragrance contained a counterbalancing proportion of one or more other fragrance components that brought the overall ΔT_{BS} of the fragrance to not more than 2.

### EXAMPLES 9 AND COMPARISONS CF

In these Examples and Comparisons, anhydrous fragranced stick compositions were prepared as summarised in Table 3 below which demonstrate the principle that interaction between gellant and fragrance likewise occurs when other gellants are employed and by suitable selection of fragrance a significantly lower ΔT_{B} can be achieved, thereby reducing the risk of instability The samples were made by the following method. They could be used as bases for an anhydrous antiperspirant or deodorant stick by the incorporation of either a conventional particulate antiperspirant active such as AZAG or AACH, or a deodorant active such as triclosan.

The carrier liquids and gellant were mixed together and heated to 85° C. and stirred until the gellant had dissolved.

The mixture was allowed to cool to about 60° C. and then poured into containers in which the calculated amo8unt of fragrance was already present, and mixed with a spatula.

TABLE 3

| | CF. 1 | Ex9.1 | CF. 2 | Ex9.2 | CF. 3 | Ex9.3 | CF. 4 |
|---|---|---|---|---|---|---|---|
| Carrier C1 | 31.36 | 31.36 | 31.36 | 31.36 | 35.28 | 35.28 | 31.36 |
| Carrier C2 | 47.04 | 47.04 | 47.04 | 47.04 | 52.92 | 52.92 | 47.04 |
| Gellant G2 | 19.60 | 19.60 | 0 | 0 | 0 | 0 | 0 |
| Gellant G3 | 0 | 0 | 19.60 | 19.60 | 0 | 0 | 0 |
| Gellant G4 | 0 | 0 | 0 | 0 | 9.80 | 9.80 | 0 |
| Gellant G1 | 0 | 0 | 0 | 0 | 0 | 0 | 19.60 |
| Fragrance CF | 2.00 | 0 | 2.00 | 0 | 2.00 | 0 | 2.00 |
| Fragrance Ex9 | 0 | 2.00 | 0 | 2.00 | 0 | 2.00 | 0 |
| total | 100 | 100 | 100 | 100 | 100 | 100 | 100 |
| ΔT_{B} | 6.3 | 0.6 | 7.3 | 1.1 | 4.1 | 1.85 | 4.6 |

Gellant G2 was beta-cellobiose 2,3,5,2′,3′,41,5′-heptanonanoate-1-cyclohexanoate made in accordance with the description in WO 02/32914.
Gellant G3 was cellulose octa(decanoate) prepared in accordance with the description in WO 00/61079
Gellant G4 is lanosterol
Fragrance CF is methoxybenzaldehyde
Fragrance Ex9 is a polycyclic aliphatic ether.

When ΔT_{BS} for comparison CF was measured, it too was about 4.6, indicating that the data obtained in an emulsion applies also to anhydrous compositions.

The shift ΔT_{B} for the Examples containing fragrance Ex9 were all significantly lower than for the comparisons containing CF.

### EXAMPLES 10/11 AND COMPARISON CG

In these Examples and comparisons, compositions were made in accordance with the process of Example 9 employing gellant G1 which contained a fragrance which had the measured or calculated ΔT_{BS} given in the Table 4 below. The stability measured in the same way as for Example 1 is also summarised in the Table.

TABLE 4

| | Dose | ΔT_{B} (at | ΔT_{BS} at | stability |
|---|---|---|---|---|
| | wt % | dose %) | 1% | (days) |
| Ex10 | 0.8 | 1.27* | 1.28 | >56 |
| CG | 1.0 | 2.08 | 2.08 | 7-14 |
| Ex11 | 0.8 | 1.17* | 1.39 | >56 |
| Ex12 | 1.0 | 1.36 | 1.36 | 21-28 |

The data given for ΔT_{B} and ΔT_{BS} is calculated from the corresponding data for fragrance components or from ΔT_{B}, except where * indicates an actual measurement.

Fragrances Ex10 to Ex12 and CG were multi component blends which had not been analysed to identify the nature and proportion of the individual components.

The fragrances in Ex10, Ex11 and Ex12, which all had a ΔT_{BS} below 2 and the dose level ΔT_{B} was below 1.5, all achieved superior product stability than did the comparison which had a ΔT_{BS} of only slightly above 2.

From Table 4, it can be seen that it is not necessary to know the precise composition of a fragrance blend in order to identify whether or not it can be employed together with a fragrance-sensitive gellant.

1. In a fragranced cosmetic composition having improved stability comprising a cosmetic active, a water-immiscible carrier for the cosmetic active, a fragrance-sensitive fibre-forming gellant for the water-immiscible carrier and a fragrance, the improvement in which the fragrance has a standard peak depression, ΔT_{BS}, of not more than 2.

2. A cosmetic composition according to claim 1 in which ΔT_{BS} of the fragrance is from 1 to 2.

3. A cosmetic composition according to claim 1 in which the fragrance has ΔT_{BS} of less than 1.5.

4. A cosmetic composition according to claim 1 in which the fragranced composition has an FF of less than 4.0, defined by
FF=ΔT_{BS x}[% W/W fragrance in the water-immiscible carrier].

5. A cosmetic composition according to claim 4 in which the composition has an FF of from 1.0 to 3.0.

6. A cosmetic composition according to claim 5 in which the fragrance has ΔT_{BS} of less than 1.5.

7. A cosmetic composition according to claim 1 in which the fibre-forming gellant comprises at least one ester linkage or is a sterol.

8. A cosmetic composition according to claim 7 in which the gellant comprises an acylated sugar.

9. A cosmetic composition according to claim 8 in which the acylated sugar is an acylated cellobiose.

10. A cosmetic composition according to claim 1 in which the water-immiscible carrier comprises a silicone oil.

11. A cosmetic composition according to claim 10 in which said fragrance-sensitive gellant is an acylated sugar.

12. A cosmetic composition according to claim 11 in which the acylated sugar is an acylated cellobiose.

13. A cosmetic composition according to claim 1 which contains a dispersion of an antiperspirant active comprising an aluminium and/or zirconium astringent salt in the water-immiscible carrier.

14. A cosmetic composition according to claim 13 in which the dispersion comprises an aqueous solution of the astringent salt.

15. A cosmetic composition according to claim 14 in which the dispersion is refractive index matched with the gelled water-immiscible carrier.

16. A cosmetic composition according to claim 13 in which said fragrance-sensitive gellant is an acylated sugar.

17. A cosmetic composition according to claim 16 in which the acylated sugar is an acylated cellobiose.

18. A cosmetic composition according to claim 17 in which the water-immiscible oil comprises a silicone oil.

19. A cosmetic composition according to claim 18 in which less than 5% w/w of the water-immiscible oil is an aliphatic or aromatic ester oil.

20. A method of making a cosmetic composition of improved stability comprising a cosmetic active, a water-immiscible carrier for the cosmetic active, a fragrance-sensitive gellant for the water-immiscible carrier and a fragrance, in which the fragrance-sensitive gellant is dissolved in the water-immiscible carrier at an elevated temperature, the resultant solution is mixed with the cosmetic active and the fragrance and filled into a dispenser or mould, the said fragrance having a peak depression, ΔT_{BS}, of not more than 2.
